(19) United States
(12) Patent Application Publication
(10) Pub. No.: US 2002/0151039 A1
(43) Pub. Date: Oct. 17, 2002

(54) DNA AMPLIFICATION USING ELECTROLYTE CONDUCTANCE HEATING AND TEMPERATURE MONITORING

(76) Inventors: CARL T. WITTWER, SALT LAKE CITY, UT (US); MARK G. HERRMANN, CLINTON, UT (US); DAVID M. HEAP, BOUNTIFUL, UT (US)

Correspondence Address: RICHARD F TRECARTIN ESQ, FLEHR HOHBACH TEST ALBRITTON & HENDERSON, FOUR EMBARCADERO CENTER SUITE 3400, SAN FRANCISCO, CA, 941114187

(21) Appl. No.: 09/373,041

(22) Filed: Aug. 12, 1999

Related U.S. Application Data
(63) Continuation-in-part of application No. 09/132,717, filed on Aug. 12, 1998, now abandoned.

(51) Int. Cl. C12M 1/40

(52) U.S. Cl. 435/286.1; 435/287.2; 435/303.1; 435/91.2; 392/323; 204/601

(57) ABSTRACT

A system for thermal cycling a sample utilizing electrolyte conductance heating. An electric current is passed through the sample to increase its temperature due to resistance heating. As the sample acquires more thermal energy its viscosity changes, which causes a change in resistance. Because of this characteristic, temperature of the sample can be measured as a function of resistance and temperature can be controlled using resistance of the solution as feedback to a circuit which controls the heating of the sample by electrical conductance.

### BACKGROUND

1. The Field of the Invention

The invention relates to apparatus and methods to carry out thermal cycling and monitoring of various biological reactions, such as the polymerase chain reaction (PCR) and ligase chain reaction (LCR).

2. The Background Art

In numerous areas of industry, technology, and research there is a need to reliably and reproducibly subject samples to thermal cycling. The need to subject a sample to repeated thermal cycles is particularly acute in biotechnology applications. In the biotechnology field, it is often desirable to repeatedly heat and cool small samples of materials over a short period of time. One such biological process that is regularly carried out is cyclic DNA amplification.

The Polymerase Chain Reaction (PCR) is one cyclic DNA amplification process by which specific sequences of DNA are amplified. The PCR cycle consists of three temperature and time dependent steps: denaturation, annealing, and polymerization. As these steps are repeated, the target sequence of DNA is amplified exponentially.

LCR is similar to PCR except that the primer probes are not extended by nucleotide additions but rather are joined by a ligase. See, Wu and Wallace (1989) Genomics 4:560-569; Weidmann et al., “Ligase Chain Reaction (LCR)—Overview and Applications” in PCR Methods and Applications, Cold Spring Harbor Laboratory (1994), 551-564. See, also EPO Publication No. 0 336 731.

While many different techniques have been used to carry out heating and cooling needed for DNA amplification, the previously available techniques all present particular disadvantages and advantages.

For example, commercial programmable metal heat blocks have been used to effect the temperature cycling of biological samples in microfuge tubes through the desired temperature versus time profile. However, the inability to quickly and accurately adjust the temperature of the heat blocks through a large temperature range over a short time period, has rendered the use of heat block type devices undesirable as a thermal cycling system when carrying out processes such as the polymerase chain reaction.

Moreover, the microfuge tubes which are used have disadvantages. The material of the microfuge tubes, their wall thickness, and the geometry of microfuge tubes is a hindrance to rapid heating and cooling of the sample contained therein. The plastic material and the thickness of the wall of microfuge tubes act as an insulator between the sample contained therein and the surrounding medium thus hindering transfer of thermal energy. Also, the geometry of the microfuge tube presents a small surface area to whatever medium is being used to transfer thermal energy.

Furthermore, devices using water baths with fluidic switching, (or mechanical transfer) have also been used as a thermal cycler for the polymerase chain reaction. Although water baths have been used in cycling a polymerase chain reaction mixture through a desired temperature versus time profile necessary for the reaction to take place, the high thermal mass of the water (and the low thermal conductivity of plastic microfuge tubes), has been significantly limiting as far as performance of the apparatus and the specificity of the reaction are concerned.

Devices using water baths are limited in their performance. This is because the water's thermal mass significantly restricts the maximum temperature versus time gradient which can be achieved thereby. Also, the water bath apparatus has been found to be very cumbersome due to the size and number of water carrying hoses and external temperature controlling devices for the water. Further the need for excessive periodic maintenance and inspection of the water fittings for the purpose of detecting leaks in a water bath apparatus is tedious and time consuming. Finally, it is difficult with the water bath apparatus to control the temperature in the sample tubes with the desired accuracy.

U.S. Pat. No. 3,616,264 to Ray shows a thermal forced air apparatus for cycling air to heat or cool biological samples to a constant temperature. Although the Ray device is somewhat effective in maintaining a constant temperature within an air chamber, it does not address the need for rapidly adjusting the temperature in a cyclical manner according to a temperature versus time profile such as is required for biological procedures such as the polymerase chain reaction.

U.S. Pat. No. 4,420,679 to Howe and U.S. Pat. No. 4,286,456 to Sisti disclose gas chromatographic ovens. The devices disclosed in the Howe and Sisti et al. patents are suited for carrying out gas chromatography procedures but do not provide thermal cycling which is substantially any more rapid than that provided by any of the earlier described devices. Rapid thermal cycling is useful for carrying out many procedures. Devices such as those described in the Howe and Sisti et al. patents are not suitable for efficiently and rapidly carrying out such reactions.

In addition, microfluidic devices have been described which are adapted for controlling the temperature in a microfluidic reaction chamber. For example, Northrop et al., U.S. Pat. Nos. 5,589,136 and 5,639,423 disclose integrated microfabricated instrumentation wherein Lamb-wave pumps are used to move fluids between reservoirs and chambers and wherein a heater is placed in close proximity to a reaction chamber. In one embodiment a layer of silicon nitride (with electrical leads) is deposited on a surface opposite a reaction chamber. An electrical current through the silicon nitride provides heat to the reaction chamber.

Wilding et al. U.S. Pat. Nos. 5,587,128 and 5,498,392 each disclose mesoscale polynucleotide amplification devices. In each case, heat is applied to a reaction chamber via a radiation source or by way of a heater located in proximity to the reaction chamber. In some microfluidic devices, electric fields are used to transport molecules, see e.g., U.S. Pat. No. 5,750,015 to Soane et al. and U.S. Pat. No. 5,858,195 to Ramsey et al

PCR and LCR are fundamental DNA amplification techniques essential to modern molecular biology. Despite their usefulness and popularity, the current understanding of PCR and LCR is not highly advanced. Amplifications must be optimized by trial and error and protocols are often followed blindly. The limited understanding of PCR and LCR found in the art is a good example of how those skilled in the art are content to utilize a powerful technique without reflection or comprehension.

Biological processes such as PCR and LCR require temperature cycling of the sample. Not only does the prior art carry out temperature cycling slowly, the prior art also ignores the underlying principles which allow PCR and LCR to work and could be used to make them even more useful. Thus, it would be a great advance in the art to provide methods and apparatus which are particularly adaptable for rapidly carrying out PCR or LCR and analyzing the reaction which is taking place, particularly if such reaction is analyzed as it is taking place, that is, in real time.

In view of the above described state of the art, the following are objects and advantages of the invention. It is an object of the present invention to provide an apparatus for accurately controlling the temperature of biological samples.

It is a further object of the invention to provide a thermal cycling apparatus for quickly and accurately varying the temperature of biological samples according to a predetermined temperature versus time profile.

It is also an object of the invention to provide a thermal cycling system wherein the sample temperature is monitored by measuring the resistance of the sample.

It is an object of the invention to provide a thermal cycling device of microfluidic dimensions where temperature is controlled by the application of a current to a reaction chamber.

It is also an object of the invention to provide a thermal cycling apparatus which can effectively subject samples to a large temperature gradient over a very short period of time.

It is an object of the invention to provide a system and method for performing PCR or LCR rapidly.

It is a further object of the invention to provide a system and method for performing PCR or LCR rapidly while also adjusting the reaction parameters while the reaction is ongoing.

It is a further object to provide a device and method for measuring the temperature of a ligase by screening the resistance of said sample.

### SUMMARY OF THE INVENTION

In accordance with the foregoing, the invention includes a thermal cycling device comprising a holder of a biological sample and first and second electrodes positioned to be in electrical contact with the biological sample. A circuit is provided for adjusting the current flow through the biological sample to regulate the temperature thereof.

In some embodiments the sample holder comprises a capillary tube whereas in others the sample holder comprises a chamber of a microfluidic device.

Heating of the biological sample occurs by electrical conductance through the sample. An alternating current is preferably applied to the biological sample to prevent electrophoretic separation of reactant components during the heating phase. As electric current is passed through the biological sample its temperature increases, due to resistance heating. As the sample acquires more thermal energy its viscosity changes, which in turn causes a change in resistance. Because of this characteristic, thermal cycling can be controlled using resistance of the sample solution as feedback to measure and control the sample temperature.

The cooling phase of a thermal cycle preferably utilizes a heat sink which can include fluids, either gaseous or liquid, at ambient or a predetermined temperature which are in contact with at least one surface of the holder of the biological sample.

The invention also includes methods and devices for measuring the temperature of a liquid sample which in some embodiments is combined with a temperature control circuit to regulate the temperature of a sample. In this aspect of the invention, a correlation between resistance and temperature is established for a particular device designed to contain a liquid sample. When the device is used for its intended purpose, the resistance across the liquid sample is measured and compared to the predetermined correlation between resistance and temperature. This provides an indication of the actual temperature of the liquid.

In an additional embodiment, the electrical resistance of the sample is used to control the sample temperature. In such embodiments, the sample resistance is compared to the resistance/temperature correlation. For a particular desired temperature, differences in the resistance are used to activate or deactivate a heat source to control the temperature of the liquid depending on whether the temperature is to be increased or decreased, respectively. In the preferred embodiment, the heat is provided to the system by way of electric conductance heating.

### BRIEF DESCRIPTION OF THE DRAWINGS

FIG. 1 is a diagrammatic representation of a preferred embodiment of the invention.

FIG. 1A is a detailed view of a portion of the system illustrated in FIG. 1.

FIG. 2 is a chart representing the temperature response obtained using the system illustrated in FIG. 1.

FIG. 3 is a is schematic representation of the electrolytic circuit formed by the system represented in FIG. 1.

FIG. 4 is a diagrammatic representation of a preferred apparatus for determining resistance dependance on temperature of a sample.

FIG. 5 is a flow chart representing a presently preferred method of the invention.

FIG. 6A is a chart showing a resistance vs. temperature plot of the biological sample.

FIG. 6B shows the relationship of resistance as a function of temperature.

FIG. 7 is a chart showing the dependance of temperature on resistance of a biological sample for the device of FIG. 1.

FIG. 8 is an electrophoretic representation showing PCR product verification using the device of FIG. 1.

FIG. 9 depicts an alternate embodiment of the invention.

FIG. 10A shows the results from a temperature-resistance calibration.

FIG. 10B depicts the time response of the resistance due to buffer cooling inside the PVDF fits of the device of FIG. 9.

FIG. 11 shows the inverse correlation of resistance to temperature during cycling.

FIGS. 12A, B and C depict the results obtained from a PCR amplification of three different regions of a hemoglobin gene.

FIGS. 13, 14, 15A and 15B depict various alternative embodiments of the invention.

### DETAILED DESCRIPTION OF THE INVENTION

The device for thermal cycling a biological sample comprises a holder for the biological sample, first and second electrodes positioned to be in electrical contact with the biological sample when present in the holder and a circuit for adjusting the current flowing through the biological sample to regulate the temperature thereof.

As used herein, a “sample holder” or “holder” of a biological sample refers to a container such as a conduit into which a biological sample is introduced. The holder is such that it can be in electrical communication with a first and second electrode which provide the current to heat the sample in the holder. While it is possible to directly insert the electrodes into the biological sample, it is preferred that the holder comprise a capillary or microfluidic channel wherein each of the ends of the capillary or channel are in fluidic contact with one or more reservoirs. Direct contact with the biological sample is not preferred due to the potential formation of gases by way of electrolysis at the electrodes which in the case of a capillary or microfluidic device could result in disruption of the electrical circuit with a concomitant increase in resistance.

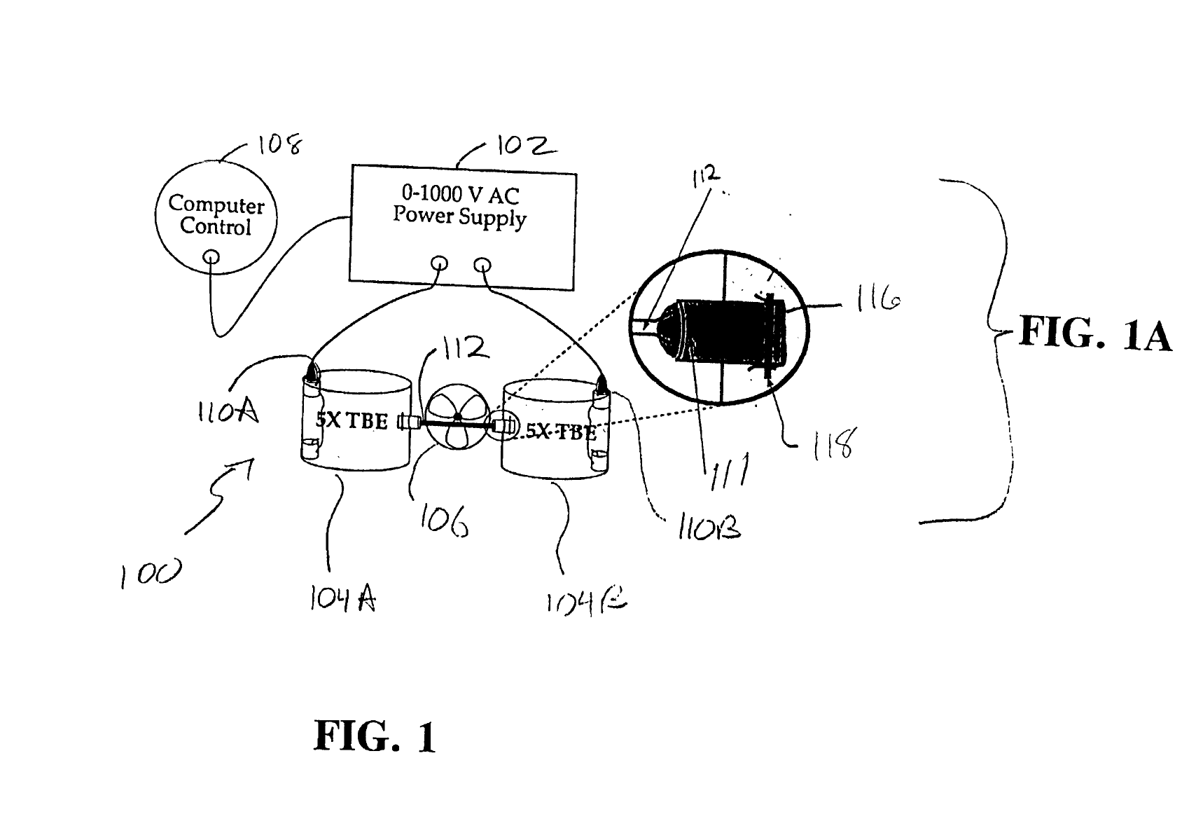

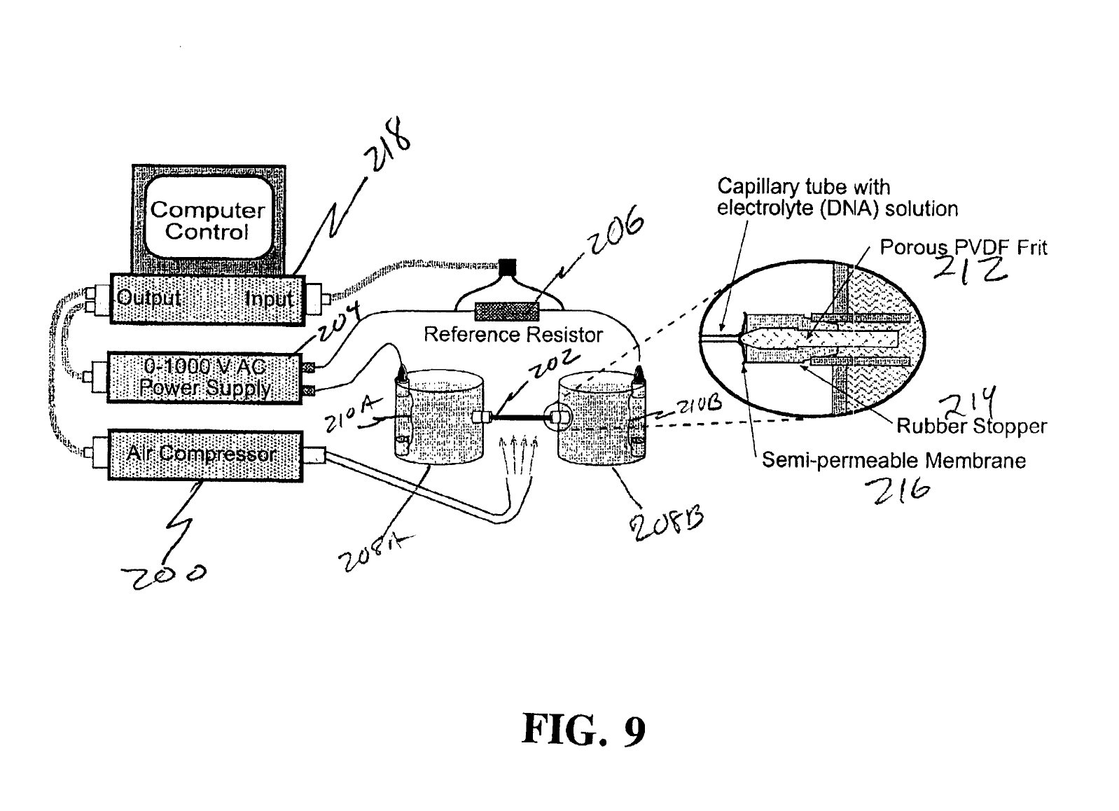

The invention is exemplified by use of capillaries such as those set forth in the Examples and in FIGS. 1 and 9. However, microfluidic devices well known to those skilled in the art can be readily modified to incorporate the subject matter of the invention. For example, U.S. Pat. Nos. 5,589,136 and 5,639,423 to Northrop et al. disclose integrated microfabricated instrumentation using silicon as a substrate and a layer of silicon nitride as a resistance source of electrical heating. Wilding et al., U.S. Pat. Nos. 5,587,128 and 5,498,392 disclose mesoscale polynucleotide amplification devices. In these patents, a reaction mixture is transported between two reaction chambers which are held at different temperatures to achieve thermal cycling. Heat for thermal cycling is applied to the reaction chambers via a radiation source or by way of heaters located in proximity to the reaction chambers. Other microfluidic devices use electric fields to transport molecules. See, e.g., U.S. Pat. Nos. 5,750,015 and 5,858,195.

The foregoing microfluidic devices as well as others known to those skilled in the art can be readily modified to provide electrodes which are in electrical communication with a reaction chamber in the microfluidic device so as to provide an electrical current between the two electrodes and through the biological sample. Such electrodes may in addition be used alone or in conjunction with other electrodes to bring about mass movement by way of electro endosmotic flow or the movement of charged molecules via electrophoresis. In this way reactants can be brought together in the sample holder and be subjected to a thermal cycling reaction such as PCR or LCR. The products produced, if any, can therefore be moved to a different region of the device for detection. In general, microfluidic devices have at least one cross sectional dimension of at least one channel or trench of between 0.1 to 1000 microns, preferably 0.5 to 500 microns, most preferably between 0.5 and 100 microns.

Included in some embodiments of the invention is a heat sink which facilitates the cooling phase of the thermal cycle. Preferred heat sinks include gaseous or liquid baths surrounding, or in contact with, an outside surface of the sample holder. Such fluids may be at ambient temperature or maintained at a predetermined temperature to facilitate a particular temperature vs. time gradient. When so used, it is preferred that the fluid be circulated to facilitate thermal exchange with the sample holder. Fluid pumps such as a fan or compressor can be used in such embodiments.

In the case of microfluidic devices, such heat transfer from a sample holder can occur by having all or the pertinent portion of the microfluidic device exposed to the heat sink. However, it is also possible to fabricate one or more channels or chambers which are in close proximity to the sample holder so as to permit rapid thermal exchange with a liquid for gas present or flowing through said channel or chamber.

The circuit for adjusting the current flowing through the biological sample to regulate the temperature thereof preferably utilizes a reference resistance in series with the sample holder. As explained in more detail hereinafter, such a circuit can be used to determine the resistance of the biological sample. As the temperature of the biological sample rises, the viscosity decreases and with it the overall resistance of the biological sample. Empirical measurement of the relationship between temperature and resistance in the biological sample in a device provides all the necessary information needed to control the temperature of the biological sample when used in conjunction with an appropriately programmed controller.

A preferred embodiment of the invention is depicted in FIG. 1. Generally represented as 100 in FIG. 1, it includes four principal components: a power supply 102, electrolytic reservoirs 104A&B, at least one cooling fan 106, and a computer controller 108. The power supply 102 is one which can be fabricated in accordance with principals known in the industry (such as one available from Industrial Test Equipment Co., Inc.) and is controlled by computer control 108 and preferably provides a single 60 Hz sine wave at 0- 1000 V rms and 0- 10 mA. It will be appreciated that power supplies having different characteristics can also be used in practicing the scope of the present invention.

An electrical circuit is made by connecting the power supply 102 to two buffer (preferably 5×TBE: 0.43M TRIS, 0.45 M Boric Acid, 1.9 mM EDTA) filled reservoirs 104A&B with an electrode 110A&B (preferably platinum) positioned in each reservoir. A capillary tube 112, preferably completely filled with the biological sample to undergo thermal cycling, is placed between the two reservoirs 104A&B and completes the electrical circuit between the electrodes 110A&B. While many different structures can be used, it is preferred that the capillary tube 112 have dimensions 1.02 mm o.d., 0.56 mm i.d., and 53.0 mm length and have flared ends. It will be appreciated that the electrodes 110A&B are not placed directly in contact with the capillary tube 112 because of electrolysis and consequent bubble formation.

Electrical contact between the capillary tube 112 and the reservoirs 104A&B is accomplished through the conductive joint represented in Figure 1A. Those skilled in the art will appreciate that useful information regarding the placement of the electrodes 110A&B can also be gleaned from capillary electrophoresis techniques. It will be appreciated that the structures represented in FIG. 1A are merely exemplary of the many different structures which can be used within the scope of the present invention and the same structure is provided on each end of the capillary tube 112.

Illustrated in FIG. 1A is an agarose gel plug 111 (1.5% agarose, 0.5×TBE: 43 mM TRIS, 45 mM Boric Acid, 0.19 mM EDTA) that is provided on the reservoir 104B ready for contact with the end of the capillary tube 112 and is held in place by a dialysis membrane 116 (for example one available from Spectrapor: No. 132655) with the dialysis membrane being held in place by a retaining O-ring 118. The fan 106, which preferably is a 115 VAC fan (for example one available from Nidec Alpha V, model A30473-10) is controlled by the computer control 108 and is used to provide forced air convection cooling of the biological sample in the capillary tube 112. It is within the scope of the invention to provide active refrigeration of the cooling airstream created by the fan 106 to increase the rate of cooling. It is preferred that the computer control 108 be programed in accordance with the invention utilizing the graphical programming language known as LabView (available from National Instruments, Austin, Tex.).

Those skilled in the art will appreciate that as an electrical current encounters resistance, energy is lost in the form of heat. This is the basis for resistance heating. The greater the electric potential across a resistor, the more energy is dissipated. The power supply 102 provides a sufficiently high voltage to the system to provide the desired heating. The majority of the electrical resistance in the circuit formed by the power supply 102, the electrodes 110 A&B, the reservoirs 104A&B, and the capillary tube 112 is due to the electrical resistance of the sample held in the capillary tube 112. As potential is applied across the sample, it heats up. If more voltage is applied, the sample heats faster and ultimately to a higher steady-state temperature. As the sample heats, its viscosity decreases, which corresponds to a decrease in resistance. Measuring the change in sample resistance is fundamental to thermal cycling control, since it is the means by which temperature is monitored in accordance with one aspect of the invention.

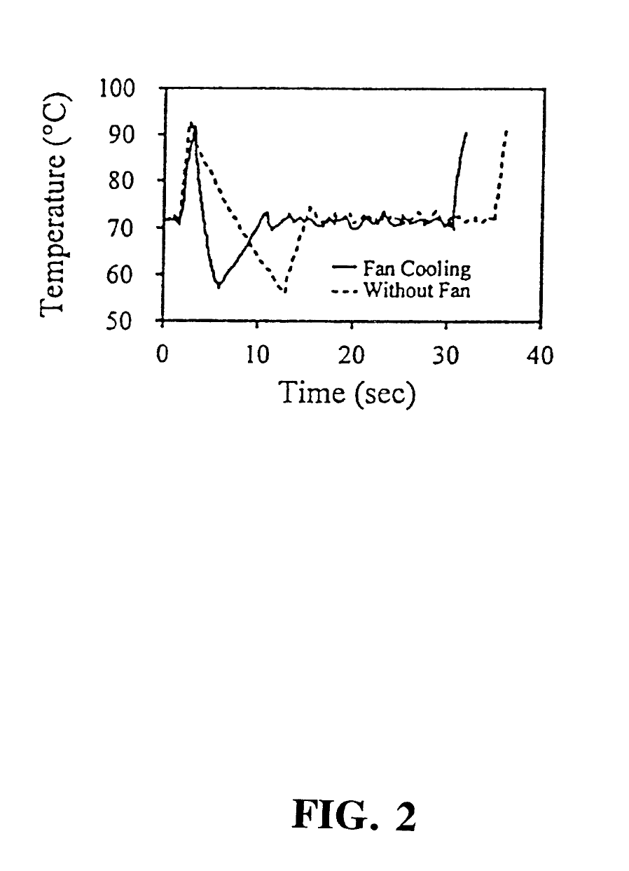

Cooling of the sample contained in the capillary tube 112 in FIG. 1 is accomplished using forced air convection. The fan 106 is positioned to move ambient air across the exterior surface of the capillary tube 112, which enhances the convection heat transfer process, as illustrated in the chart of FIG. 2.

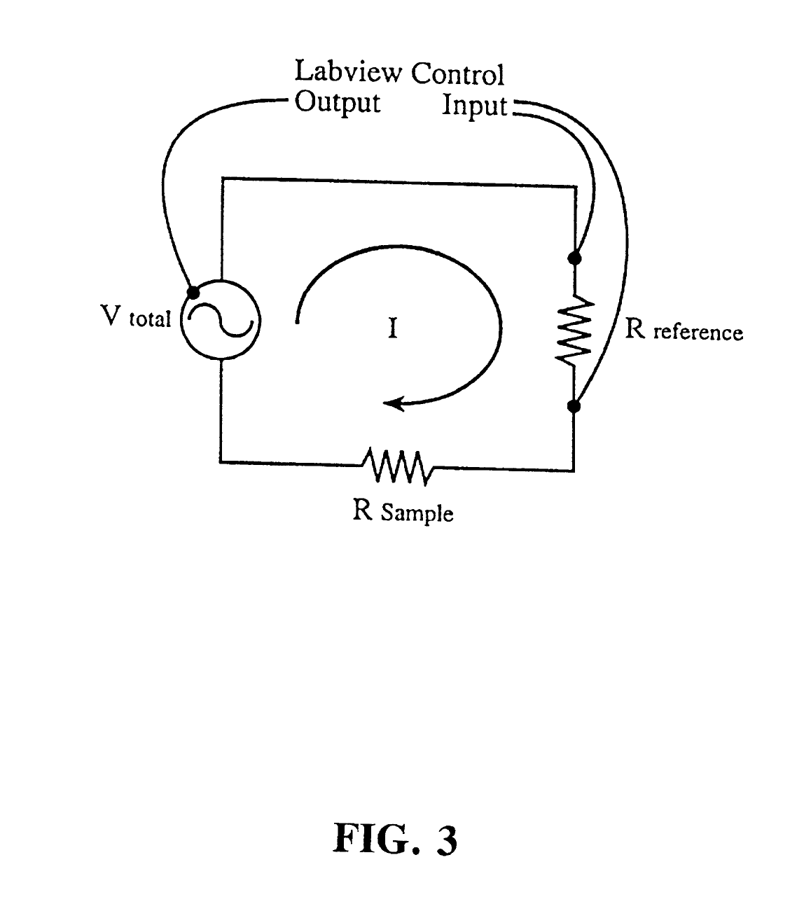

To determine the resistance of the sample, a simple voltage divider is made by placing a known reference resistor in series with the sample as shown in FIG. 3. If the potential drop across the fixed resistor is measured, the current can then be calculated using Ohm's law. Since the reference resistance is in series with the sample, the same current passes through each resistance. The total resistance of the system is the sum of the reference resistance and the sample resistance.

Rₜₒₜₐₗ=R_{reference}+Rₛₐₘₚₗₑ Equation (1)

Described by Ohm's law, the total resistance is equal to the total voltage generated by the power supply divided by the current. Substituting this into the previous equation and solving for the resistance of the sample yields:

$${R}_{sample} = {R}_{reference} ( \frac{{V}_{total}}{{V}_{reference}} - 1 ) \qquad Equation ( 2 )$$

R_{reference} is known and Vₜₒₜₐₗ and V_{reference} can be measured, so Rₛₐₘₚₗₑ can be calculated.

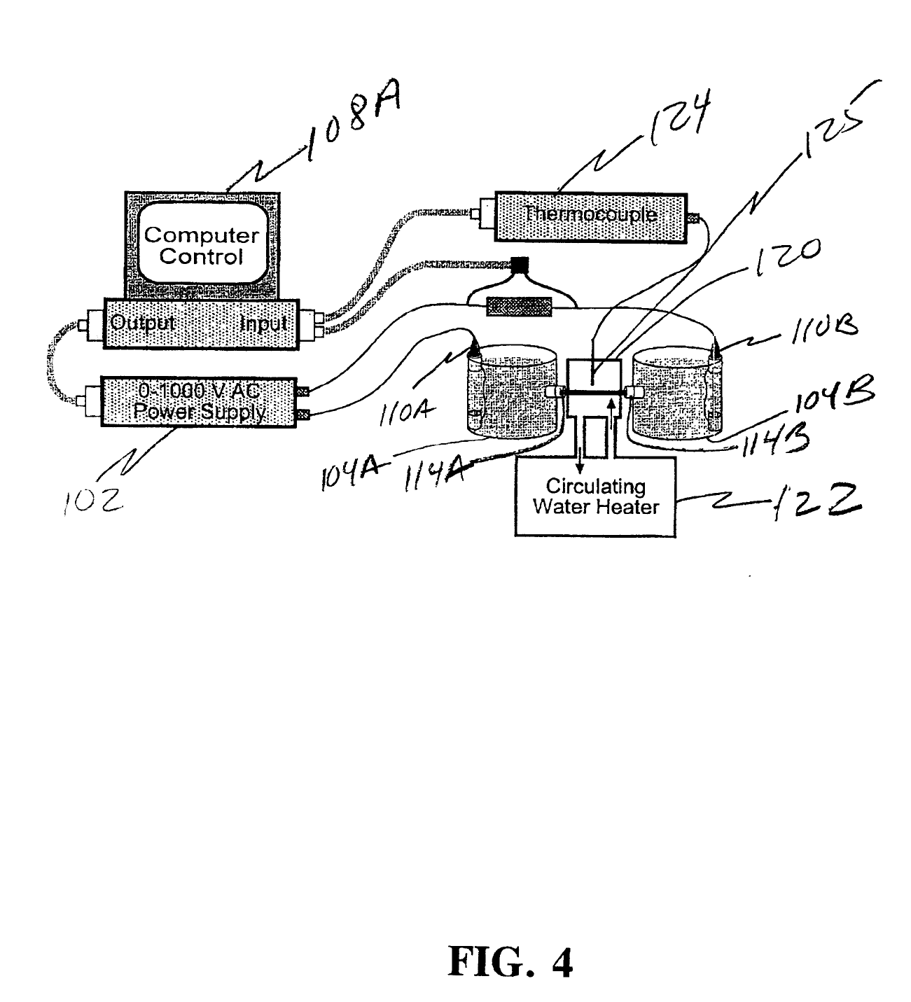

Reference will next be made to FIG. 4 which is a diagrammatic representation of a preferred apparatus for determining the resistance dependance on temperature of a sample. In accordance with the present invention, the correlation between resistance of the sample and temperature of the sample is determined by placing the capillary tube inside a water filled chamber 120 as shown in FIG. 4. A water heater/pump 122, for example one available from Haake E1, number 000-5708 which heats the water at 0.1° C./sec, is used to circulate the water through the chamber 120. While the water in the chamber is heated a small amount of current is passed through the sample, so that its resistance can be measured. Because the temperature of the water changes slowly, the sample in the capillary tube is in thermal equilibrium with the water surrounding it in the chamber 120. It will be appreciated that many structures other than the water filled chamber 120 can be used, for example the chamber 120 may be filled with another fluid.

In practice, the difference between the sample temperature and chamber temperature has been measured to be less than 1° C. The potential across the sample must be small enough that heating is insignificant compared to thermal equilibrium with the water bath. Water temperature is measured, preferably with a type T thermocouple 124 (time constant 0.005 seconds) using a thermocouple thermometer 125 (for example, Physitemp, BAT-10) and the sample resistance is calculated using the above provided equation. The water temperature and the sample resistance are monitored (30000 points/sec) simultaneously by a data acquisition program, preferably written in LabView. This data is then analyzed by a least square curve-fitting scheme to yield an equation for the resistance as a function of temperature.

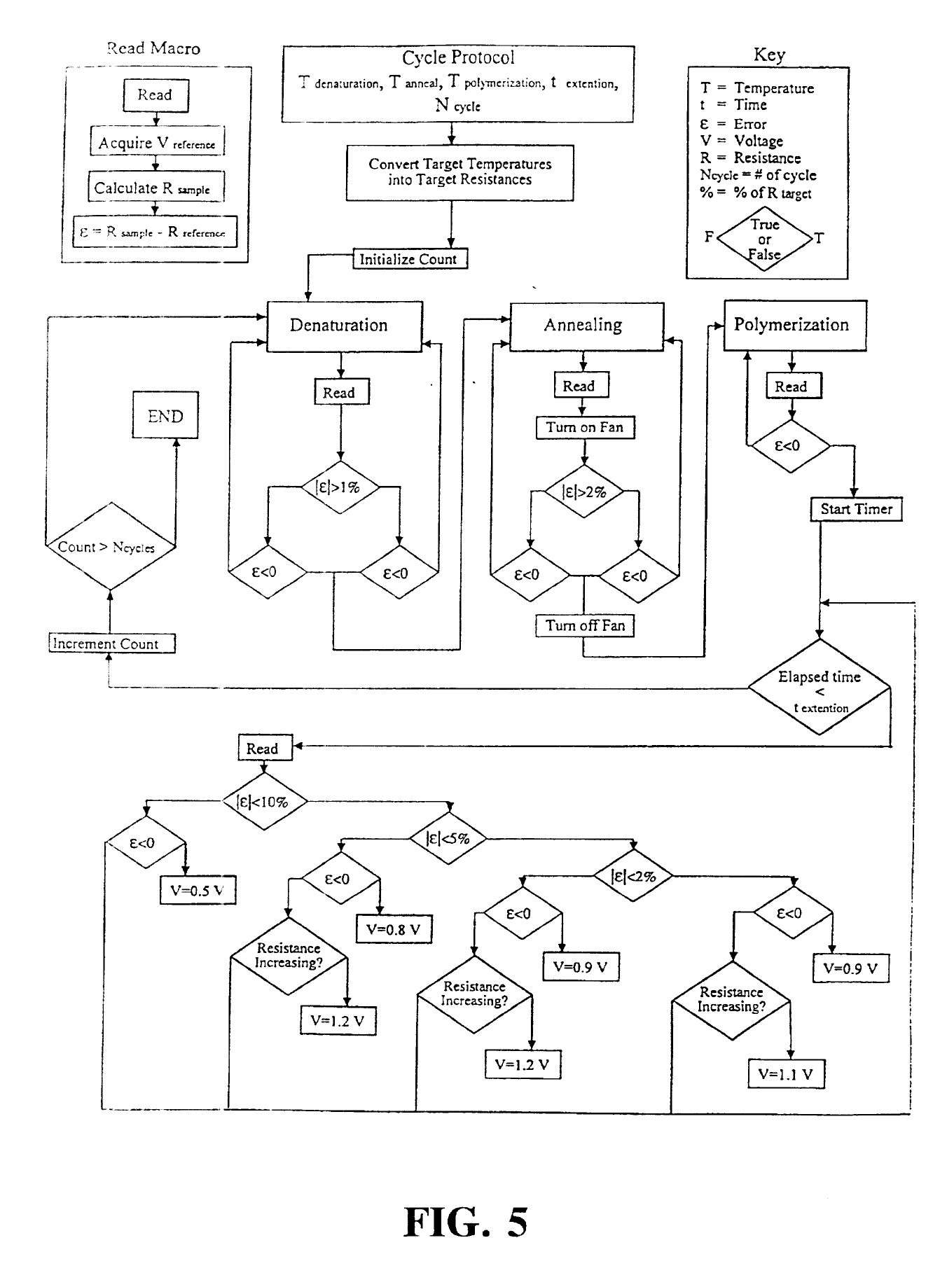

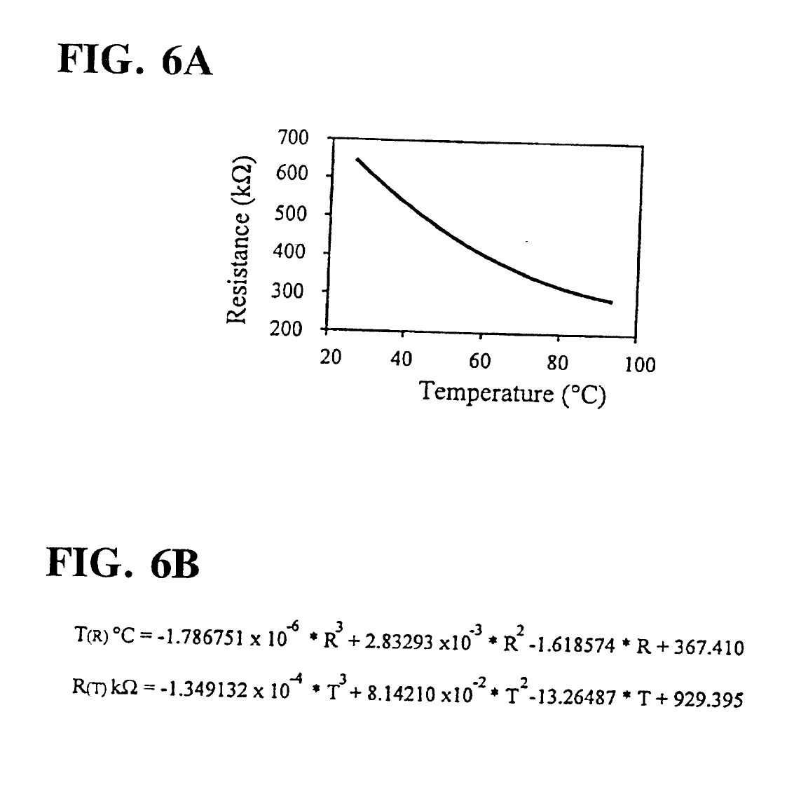

A flow chart showing one preferred method of the present invention is shown in FIG. 5. In particular, the method represented in FIG. 5 is carried out by the control structures described herein. A graphic representation of the relationship between sample resistance and temperature is shown in FIG. 6A. The equation for the resistance as a function of temperature is used to convert the denaturing, annealing and polymerization temperatures into target resistances (see B in FIG. 6). The preferred control program continuously monitors (preferably 30,000 points/sec) the sample resistance and uses it as feedback. This resistance is compared to the target resistance so that the program can properly control the power supply voltage (102 in FIG. 1) and the cooling fan (106 in FIG. 1). It will be appreciated that monitoring the resistance of the sample can be carried out in different ways, for example alternating the high voltage heating current with a low voltage conductance signal or a regulated +/−5 VDC signal can be introduced as the conductance signal for a fraction of each half cycle of the sine wave provided by the power supply (102 in FIG. 1).

Another apparatus used for thermal cycling is depicted in FIG. 9. Cooling is provided when needed by a computer controlled air compressor 200 that blows ambient air onto the capillary tube 202. A custom power supply 204 by Industrial Test Equipment Co., Inc. (Port Washington, N.Y., USA) provides a single 60 Hz sine wave at 0-1000 V rms. The reference resistor 206 has a fixed resistance of 1 kΩ and is rated at 2 Watts. The buffer reservoirs are made of acrylic tubes. The electrical leads from the power supply 208A and 208B are connected to platinum electrodes 210A and 210B in the reservoirs via banana jacks. The reservoirs are filled with approximately 250 mL of buffer (50 mM Tris-HCL, pH 8.3 (25° C.) and 3 mM MgCl₂). The DNA sample to be amplified is contained within a glass capillary tube 50 mm×0.56 mm ID) with flared ends. The capillary tube 202 is held between the two buffer reservoirs. The interface between the capillary tube and the reservoirs which is a conductive joint which includes a semipermeable membrane is shown in the exploded section view of FIG. 9. A porous PVDF (polyvinylidene fluoride) frit 212 by Porex Technologies (Fairburn, Ga., USA) is used as a bridge for electrical conductance, and is held in place with a rubber stopper 214. A semi-permeable membrane 216 (molecular weight cut-off of 100 Daltons) by Spectrum Laboratories, Inc. (Laguna Hills, Calif., USA), is placed between the sample and the PVDF frits to contain all of the reagents inside the capillary tube, while allowing ion transfer with the buffer in the reservoirs.

The apparatus is controlled using a 12-bit input/output data acquisition board 218 and software written in LabView (National Instruments, Austin, Tex., USA).

Temperature monitoring and control by electrolyte resistance is especially useful when (1) rapid changes are desired, (2) small samples are used, and (3) if electronic control is desired. PCR amplifications “on a chip” can both heat and measure temperature using only simple electrical resistance, and variety of sample geometry configurations can be imagined.

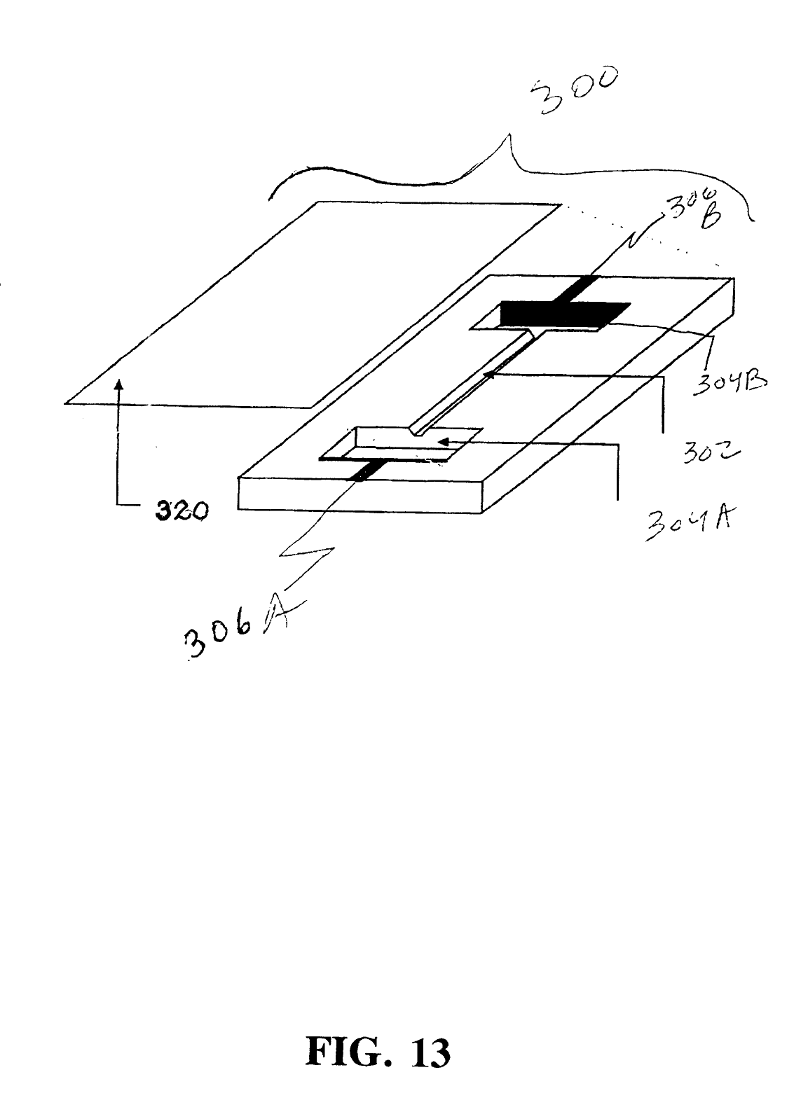

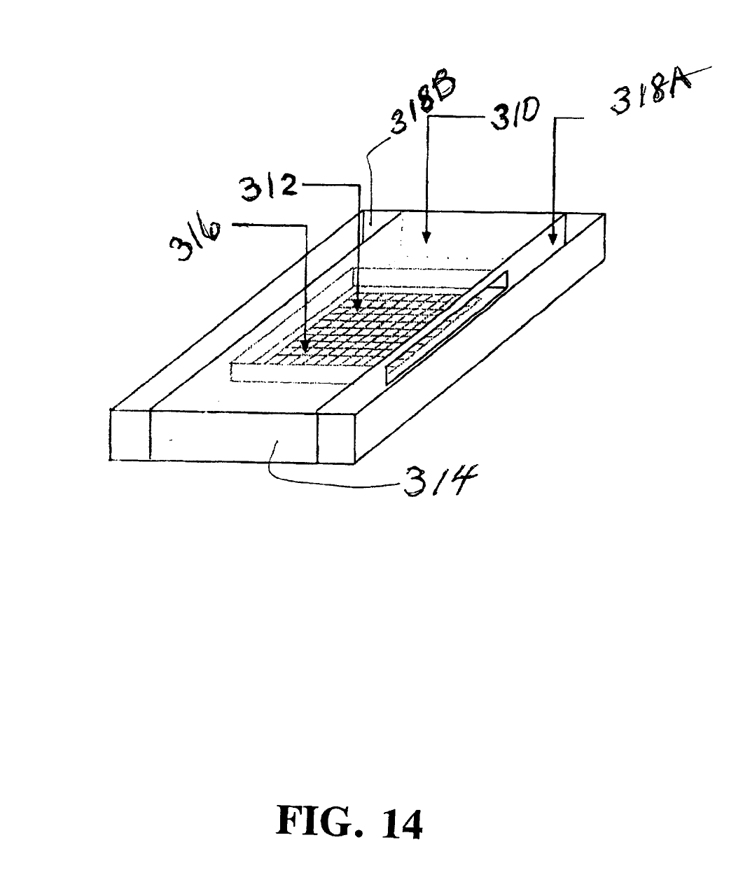

For example, etching of silicon or glass can provide a microfluidic device 300 containing long, thin channels 302 as the sample containers for the electrolyte solution (FIG. 13). The channels can be terminated in reservoir wells 304A and 304B where electrodes 306A and 306B are placed. Cover 320 isolates the channel and reservoir but not across to electrodes 306A and 306B. The sample solution and/or DNA template can be transferred into and out of the channels 302 by bulk fluid flow, or by electrokinetic or electro-endosmotic flow. Alternatively, a sample space resembling a thin wafer (square or rectangular sheet) can be etched or fabricated with electrodes or buffer reservoirs along opposite edges of the wafer (See, FIG. 14). Many sample compartments can be placed on the same substrate for multiplex or sequential amplification and analysis. Compartment surfaces may consist of optically clear windows 310 for real-time monitoring. Oligonucleotide probes may be immobilized in a grid pattern 312 on the substrate 314 in sample chamber 316 and exposed to the sample for hybridization analysis. Reservoirs 318A and 318B are in fluid communication with chamber 316. Electrodes can be formed in the reservoir or inserted into the reservoir before use.

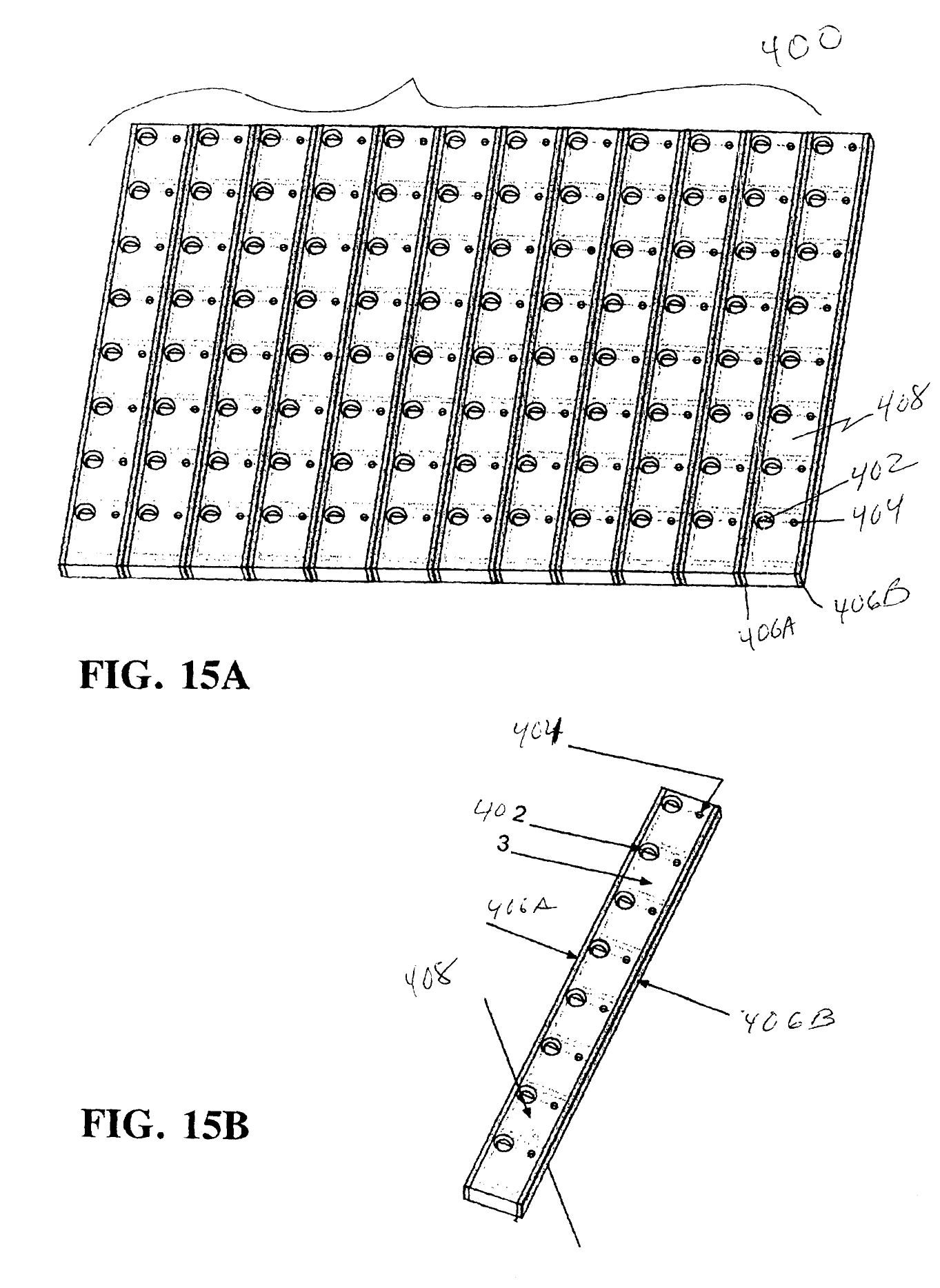

Another configuration for temperature cycler that uses electrolytic temperature measurement and heating is configured in a 96 well microtiter format (See, FIG. 15A). A multicell column which can be used to fabricate the 96 well embodiment is shown in FIG. 15B. Using any standard automated 96 well pipetting system, the samples are loaded through the hoes 402 in the upper corner of each cell. Vent holes 404 are provided to allow the air in the cell to evacuate as the sample is introduced. The cells are then brought into an upright position with the vent holes towards the top. This allows any bubbles formed during the loading or cycling processes to float to the top of each cell.

The rectangular cells are sandwiched between two long thin electrodes 406A and 406B. The same electrical potential is applied to each column of cells with each column being independently controlled. However, it is possible to modify the electrodes such that each cell is capable of independent temperature monitoring and control. AC current is applied through the width of the sample for heating and temperature monitoring. Cooling is by conduction through the cell walls. Real-time fluorescent detection is achieved by viewing through the depth of the optically clear sample cells 408. The 96 well format allows standardized sample handling and convenient real-time fluorescent monitoring.

Presently preferred examples of the method carried out using the present invention will now be described with the understanding that these examples are not intended to be limiting of the invention but to be merely exemplary of the scope of the invention.

### EXAMPLE 1

This example uses the device as set forth in FIG. 1.

A PCR reaction was performed using the following materials and procedures: Each 20 μL PCR reaction contains, 50 mM Tris-HCl, pH 8.5 (25° C.), 3 mM MgCl2, 500 μg/mL bovine serum albumin, 0.5 μM of each primer, 0.2 mM of each deoxyribonucleoside triphosphate and 0.8 U of Taq DNA polymerase per 20 μL sample. Human genomic DNA that was denatured for 10 minutes by boiling was used as DNA template for all experiments at 0.5 ng/μL. The primers that were used were PCO3 5′-ACACAACTGTGTTCACTAGC-3′ and PCO4 5′-CAACTTCATCCACGTTCACC-3′. This primer set amplifies a 110 bp region of the Human β-globin gene, according to the following protocol: denaturation at 94° C. for 0 seconds, annealing at 55° C. for 0 seconds, polymerization at 72° C. for 7 seconds.

A 50 μL sample is made, so that there is enough for two 20 μL reactions and a 10 μL control reaction. The sample mixture is then degassed in a vacuum for 20 minutes. This prevents bubble formation inside the capillary tube that otherwise will break the electrical circuit by stopping the current flow.

The structure described herein is able to provide thermal cycling at rates preferably at least as great as at 5° C./sec., more preferably at least as great as 10° C./sec., and most preferably at least as great as 20° C./sec.

The calibration of temperature to resistance for the sample is carried out next. This is done by loading the sample into a capillary tube surrounded by a water bath chamber (120 in FIG. 4), then pressing the reservoir's gel plugs (114 in FIG. 1) up against each end of the capillary tube (see FIG. 1A) to complete the electrical circuit. Fresh 1.5× agarose gel should be used to make the connection. The water bath should initially be at a temperature lower than the lowest temperature in the cycling parameters. The water heater/pump and power supply are turned on, and the data acquisition is initiated. The computer control and monitoring (108A in FIG. 4) continues to monitor the water temperature and sample resistance until the water boils. After completion, the data is analyzed to obtain an equation for resistance as a function of temperature as described previously.

The cycling protocol and the resistance equation are input to the control program. The cycle protocol consists of the denaturation, annealing, and extension temperatures and holding times as well as the number of cycles to be completed. A new capillary tube is filled with sample and placed between the gel plugs (114A&B in FIG. 4) on the reservoirs (104A&B in FIG. 4). The capillary tube should be filled so that a bead of sample is visible at both ends and so that it will form an airtight connection with the gel plug and reservoir (see FIG. 1A). Also, it is preferred that the same gel plugs should be used for this reaction as were used for the procedures carried out with the water bath in place, to ensure that differences in electrical resistance are not introduced by different gel plugs.

With the appropriate structures in place, the cycling program is run. As the resistance of the sample approaches the target resistance, the control program adjusts the power supply voltage to avoid overshoot. For example, to denature the sample, 1000 VAC is initially applied for a fast heating ramp to about 94° C. For annealing the target annealing temperature is 55° C. and 100 VAC is applied so that it does not significantly heat the sample, but allows the resistance to be monitored. During this step the electric fan is also turned on, to enhance the convection cooling process. For polymerization the temperature is increased by applying 700 volts. Once the temperature reaches 72° C., it is preferred that the voltage is adjusted so that the temperature can be maintained for seven seconds to allow for product extension. The procedure is repeated for a total of 35 cycles.

The remaining 10 μL of sample in this example is used as a control. The remaining 10 μL of sample is loaded into a glass capillary tube (1.02 mm o.d., 0.56 mm i.d., and 108.0 mm length) which is then sealed and placed into a Rapid Cycler™ thermal cycler (available from Idaho Technology or Idaho Falls, Id.). See, U.S. Pat. No. 5,455,175 and PCT Publication WO 97/46707. The same cycling protocol was used to run the control sample as well as the test sample.

When the PCR cycling is completed, the amplified samples are removed from the capillary tubes and loading buffer is added. The samples are then loaded onto an electrophoresis gel for product visualization.

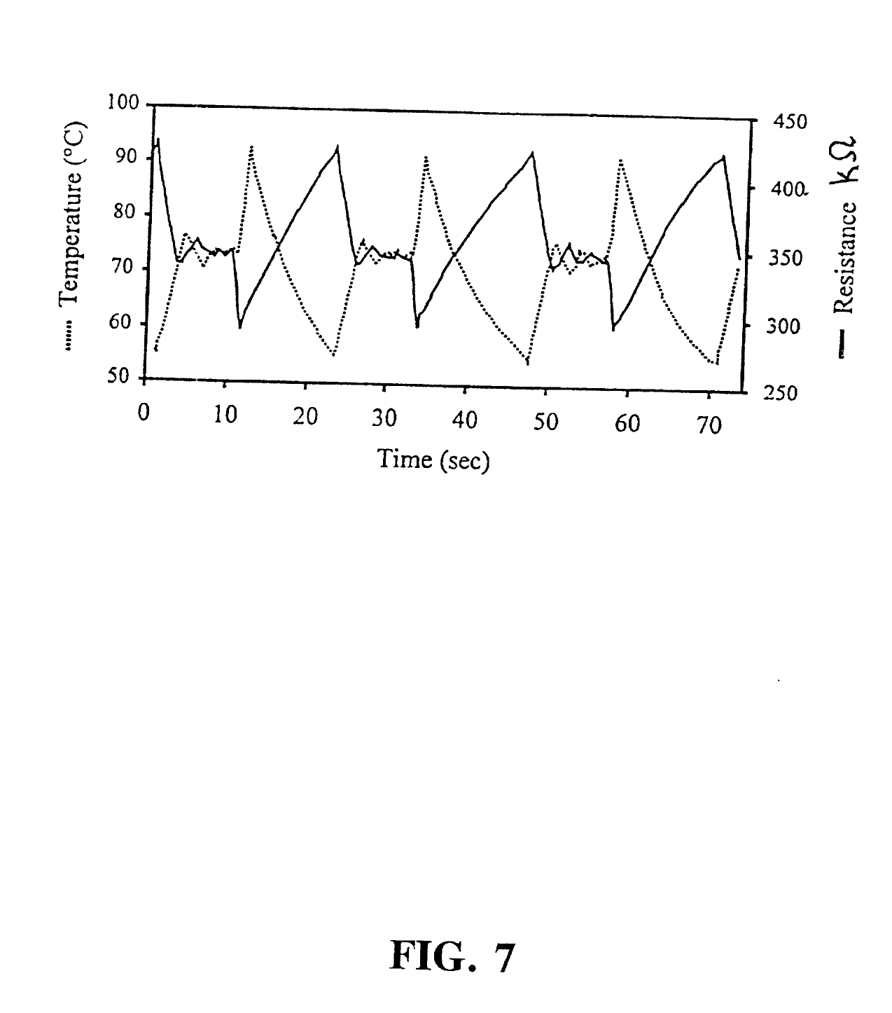

FIG. 6 (see portion A) shows the correlation of resistance with temperature as obtained using the water bath procedure. The equations for temperature as a function of resistance and resistance as a function of temperature were calculated from this correlation (see portion B of FIG. 6). FIG. 7 shows a plot of the sample resistance versus time as well as the sample temperature versus time. The sample temperature was computed using the equation for temperature as a function of resistance.

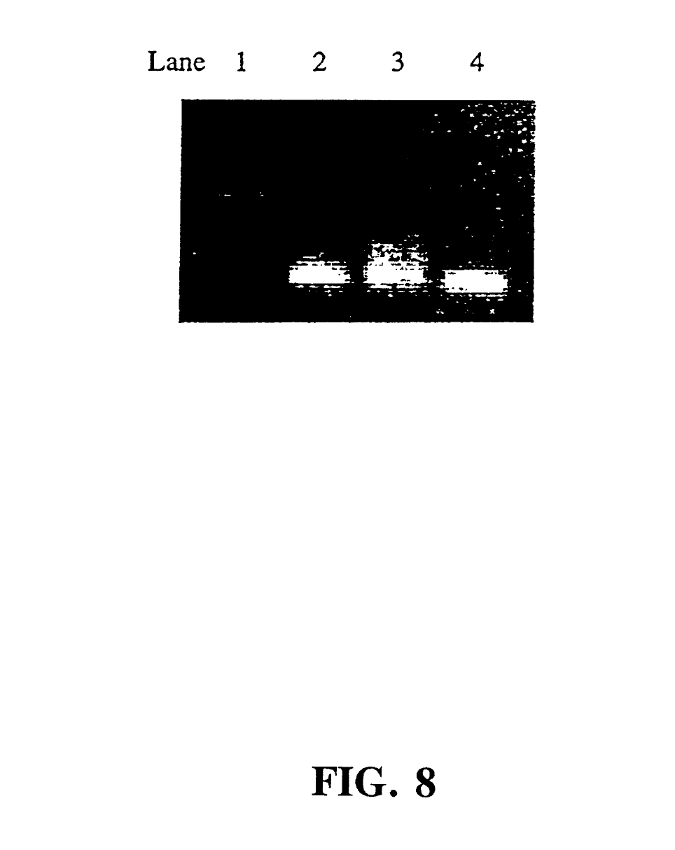

Two test samples were amplified using the presently preferred system and one control sample was amplified using the RAPID CYCLER™ thermal cycler. The amplified samples were then placed on an electrophoresis gel for product visualization as shown in FIG. 8. In FIG. 8, lane 1 is a molecular weight marker. Lanes 2 and 3 were both run using the presently preferred system but using different cycle protocols (lane 2: denaturation at 94° C. for 0 seconds, annealing at 54° C. for 0 seconds, polymerization at 72° C. for 7 seconds, lane 3: denaturation at 94° C. for 0 seconds, annealing at 56° C. for 0 seconds, polymerization at 73° C. for 7 seconds). Lane 4 was the control performed on the RAPID CYCLER™ thermal cycler (denaturation at 94° C. for 0 seconds, annealing at 54° C. for 0 seconds, polymerization at 72° C. for 7 seconds). All of the PCR samples show evidence of amplified product of the expected 110 bp length.

### EXAMPLE 2

This example utilizes the device as set forth in FIG. 9.

To control the sample's temperature a water bath enclosing the capillary tube was constructed (See, FIG. 4). A type T thermocouple and digital thermometer (Physitemp Instruments, Inc., Clifton N.J., USA) were used to measure the water temperature controlled by a circulator (Haake, Karlsruhe, Germany).

PCR was performed as follows: Each 15 μL sample to be temperature cycled contained, 50 mM Tris-HCL, pH 8.5 (25° C.), 3 MM MgCl_{2,} 500 μg/mL bovine serum albumin, 0.5 μM of each primer, 0.2 mM of each deoxyribonucleoside triphosphate, 0.375 units of Taq DNA polymerase (Boehringer Manheim, Germany) and 55 ng of Taqstart™ antibody (CloneTech, Palo Alto, Calif., USA).

Human genomic DNA, denatured for 5 minutes by boiling, was used as the DNA template for all experiments at 75 ng. Three primer sets were used to amplify different length regiouns of β-globin gene.

| | |
|---|---|
| 110 bp region: | Forward 5′-ACA CAA CTG TGT TCA CTA GC-3′ |
| | Reverse 5′-CAA CTT CAT CCA CGT TCA CC-3′ |
| 214 bp region: | Forward 5′-AGT CAG GGC AGA GCC ATC TA-3′ |
| | Reverse 5′-GTT TCT ATT GGT CTC CTT AAA CCT G-3′ |
| 536 bp region: | Forward 5′-GGT TGG CCA ATC TAC TCC CAG G-3′ |
| | Reverse 5′-GCT CAC TCA GTG TGG CAA AG-3′ |

The final sample solution was degassed in a vacuum for approximately 30 minutes to prevent bubble formation.

The following protocol was used for product amplification: For the 110 bp region; denaturation at 94° C. for 0 seconds, annealing at 55° C. for 0 seconds, polymerization at 72° C. for 1 second and a cycle time of 16.0 seconds. For the 214 bp region; denaturation at 94° C. for 0 seconds, annealing at 55° C. for 0 seconds, polymerization at 72° C. for 5 seconds and a cycle time of 20.0 seconds. For the 500 bp region; denaturation at 94° C. for 0 seconds, annealing at 55° C. for 0 seconds, polymerization at 74° C. for 20 seconds and a cycle time of 32.2 seconds.

Rₜₒₜₐₗ is the resistance of the load between the platinum electrodes. The load can be divided into three resistive components, the sample solution in the capillary tube, the buffer in the reservoirs, and the buffer inside the PVDF frits.

The correlation of sample temperature to resistance is critical for electrolytic temperature control. The goal of the calibration procedure is to establish a standard curve relating the sample's temperature to its resistance. During cycling, both the sample in the capillary tube and the buffer inside the PVDF frits heat. However, they do not necessarily heat at the same rates. Therefore, the change in measured resistance during cycling can be reviewed as the sum of the changes of the sample resistance and the PVDF frit resistance. The buffer in the reservoir does not experience a significant change in temperature during cycling, so its contribution to the load resistance remains constant.

The first step in calibration is to measure the load resistance over a range of sample temperatures while holding the temperature of the PVDF frits constant. A water bath, that completely surrounds the capillary tube, is used to control the sample temperature in a manner similar to that depiced in FIG. 4. A relativelty low voltage (90 VAC) is applied so that the load resistance can be measured without significantly heating the buffer in the PVDF frits. The sample is kept at thermal equilibrium with the water bath. The water temperature and the load resistance are monitored continuously while the water is slowly heated from ambient temperature to about 95° C. The resistance data is plotted against the temperature data and a third order polynormial equation is fit to this curve by least squares. The effect of slight changes in tube length and reagent variations are corrected by proporational adjustment of the calibration curve just prior to cycling. The sample is thermally equilibrated to ambient temperature and the load resistance is measured at low voltage (90 VAC). The temperature of the sample and the load resistance are used to determine the proportional offset.

The second step of the calibration is to hold the sample temperature constant while varying the buffer temperature inside the PVDF frits. This isolates the temperature effect of the frit resistance. The water bath (FIG. 4) is used to keep the temperature of the sample constant while the temperature of the buffer changes. Practically all of the buffer heating occurs inside the PVDF frits and the time response of the resistance is measured. The water bath is held at 70° C. and the applied voltage at 1000 VAC to achieve equilibrium and heat up the buffer in the frits. Then the voltage is stepped down to 90 VAC and the resistance time response of the resistance is measured as the frits cool. This same process is repeated for initial voltages of 200-1000 VAC to study the entire range of frit temperatures. From this daa, equations for the change in frit resistance as a function of time can be determined for heating and cooling.

$$Δ {R}_{frits} = {\underbrace{- δ exp ( - t / τ )}}_{heating} , {\underbrace{δ [ 1 - exp ( - t / τ )}}_{cooling} \qquad Equation ( 3 )$$

δ is the total change in resistance defined as the difference between the maximum and the minimum resistance, t is the time in seconds where t=0 when cooling or heating begins, and τ is the time constant in seconds.

Equation (1) can now be rearranged to express the following:

$${R}_{sample} = {R}_{ref} ( \frac{{V}_{tot}}{{V}_{ref} - 1} ) - Δ {R}_{frits} \qquad Equation ( 4 )$$

Where Rₛₐₘₚₗₑ is the combined resistance of the sample and the constant reservoir buffer resistance, and ΔR_{frits} is calculated from Equation 3.

Thermal Cycling

Cycling is accomplished using the setup depicted in FIG. 9. The protocol used by the temperature control software consists of the target temperatures for each step, the calculated proportional adjustment, the extension hold times and the total number of cycles to be completed. The software then calculates target resistances based on the standard curve, and thermally cycles the sample by using the sample resistance (Equation 4) as feedback to control the power supply voltage. In general, the sample is heated by applying 1000 VAC from the power supply. During cooling, the voltage is stepped down to 90 VAC and compressed air is blown across the capillary tube to enhance cooling by convection. To perform a temperature hold, the applied voltage is adjusted using a proportional control algorithm to maintain the sample temperature as desired.

When the PCR cycling is completed, the amplified samples are removed from the capillary tubes and the samples are loaded onto a 1.5% agarose gel for product visualization with 0.5 μg/ml ethidium bromide and UV transillumination.

The major safety risk posed by this setup is electric shock. The power supply generates enough current and voltage to cause sever shock and possible electrocution. Extreme caution must be used when setting up experiments, especially when handling the buffer reservoirs or while loading the sample in the capillary tube. During setup or when not in use, the power supply must be turned off.

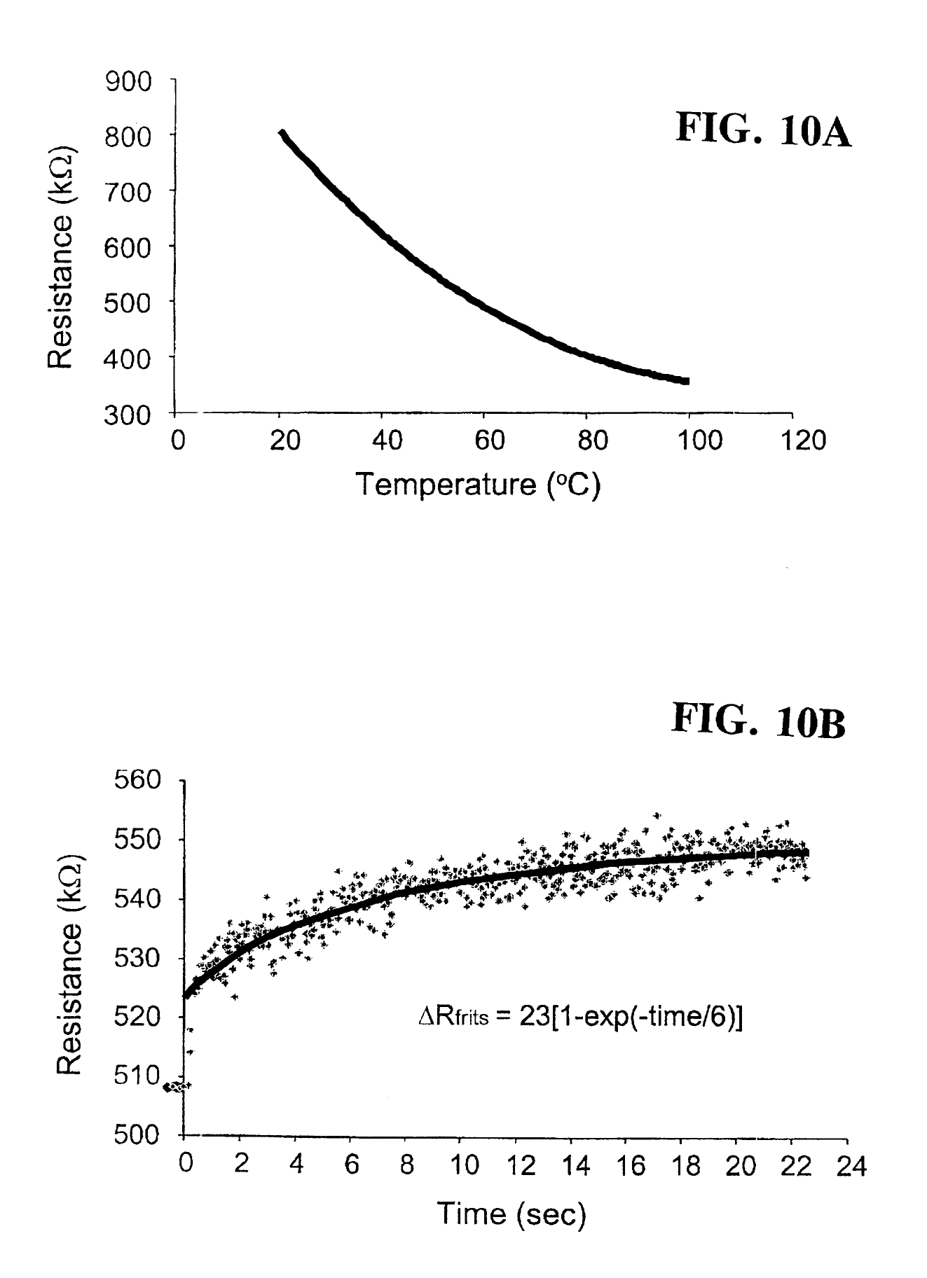

The above mentioned method for calculating the load resistance makes a fundamental assumpetion that the electrolytic solution and buffer obey ohms law. In general, this is not the case. Polarization layers established within the solution add a capacitive component to the measured impedance of the load, thus making the measured resistance afunction of applied voltage. However, by using an alternating current, the formation of the polarization layers is impeded and the capacitance can be neglected. See, e.g., Ehrhardt, W. C., IR Drop in Electrochemical Corrosion Sutdies—Part 2: A Multiple Method IR Compensation System; The Measurement and Correciton of Electrolyte Resistance in Electrochemical Tests, ASTMSTP 1056; L. L. Scribner, S. R. Taylor, Eds., American Society for Testing and Materials, Philadelphia, 1990, 78-94. Alternating current was also employed to prevent bulk electrophoresis. FIG. 10A shows the results from a temperature-resistance calibration. The inverse relationship between temperature and resistance is clearly depicted by the standard curve in FIG. 10A, which was determined using the water bath to change the sample temperature and holding the frit temperature constant. As the temperature of the sample solution increases, the resistance of the system decreases. This trend should be anticipated since at higher temperatures the viscosity of an electrolytic solution decreases, which leads to higher ion mobility, and thus lower resistance.

FIG. 10B depicts the time response of the resistance due to buffer cooling inside the PVDF fits of the device of FIG. 9. Time zero is defined as the time at which the applied voltage was stepped down to 90 volts. There are two distinct regions of the plot. The first region is the dramatic increase of resistance immediately following the step down in voltage. This increase in voltage is due to a rapid change in the sample temperature inside the capillary. Initially, the applied voltage is relatively high and some sample heating occurs even in the presence of the water bath. However, as soon as the applied voltage is dropped to 90 VAC the sample quickly cools to the temperature of the water surrounding it, which corresponds to a rapid increase in resistance. The second region of the plot characterizes the cooling rate of the buffer inside the frits. An exponential equation fit to this second region determines the coefficient and time constant for Equation (4). The time constant for heating and cooling by conduction are the same.

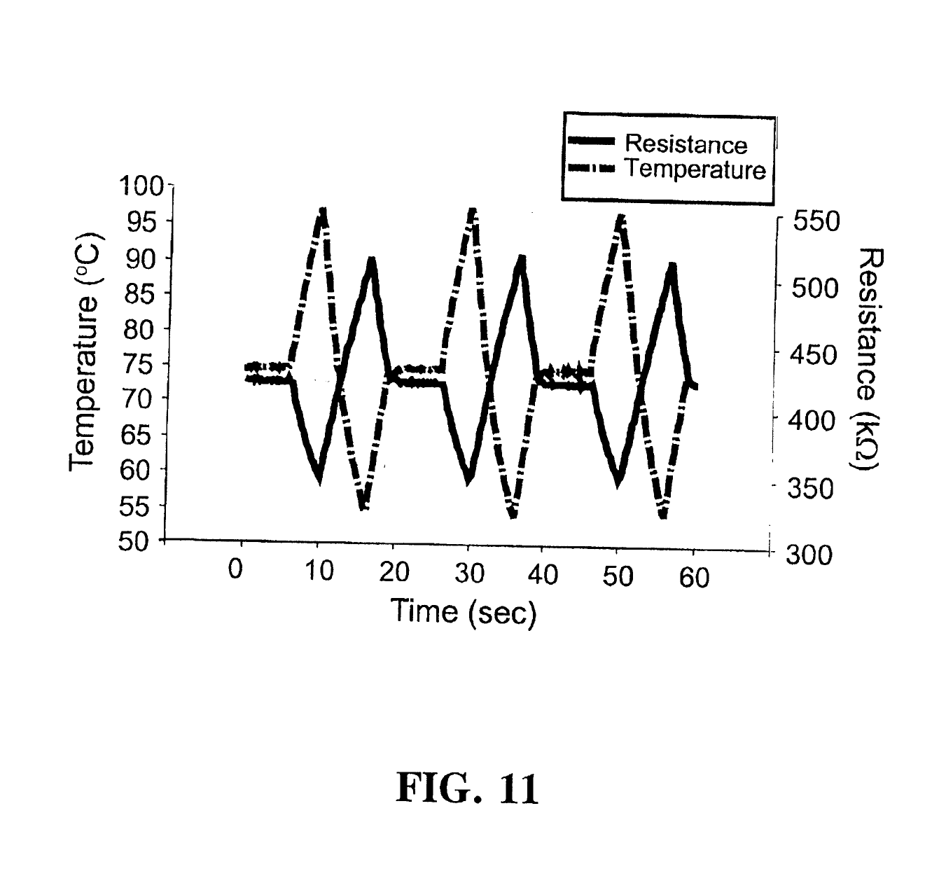

FIG. 11 shows the inverse correlation of resistance to temperature during cycling. The cuycling protocol was 94° C. denature, 55° C. anneal, 72° C. extend for 7 seconds, with a cycle time of 20 seconds.

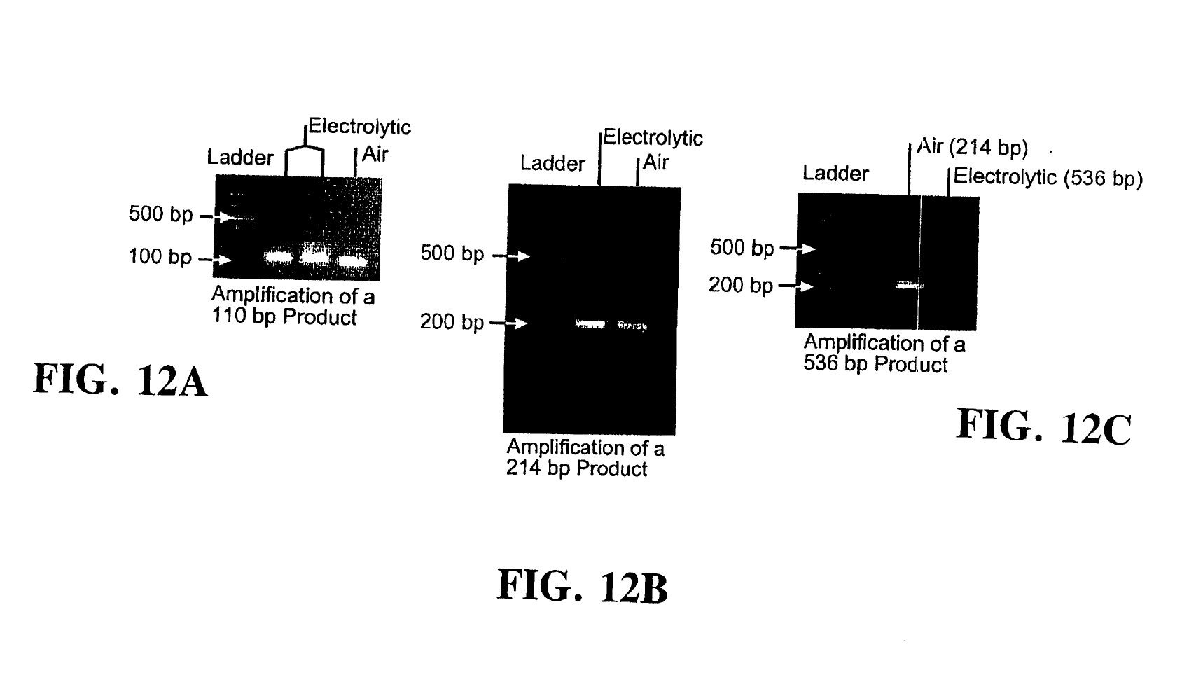

Three different regions of the hemoglobin gene of length 110 bp, 214 bp and 536 bp were amplified and visualized on electrophoretic gels (FIGS. 12A, B and C respectively). Positive controls for each experiment were amplified using a commercially available air thermal cycler from Idaho Technology (Idaho Falls, Id., USA).

From the forgoing, it has been demonstrated that rapid temperature control and PCR amplification is practical using direct Joule heating. It will be appreciated that the invention provides the most efficient heating procedure possible as the biological sample is directly heated. It will be understood that the geometry of the structure holding the sample need not be tubular, although it is preferred that the cross sectional area of the sample container should be kept constant between electrodes to ensure uniform conductance through the sample. Moreover, it is within the scope of the present invention to combine a plurality of sample holding structures to simultaneously carry out thermal cycling on a plurality of samples.

It will be appreciated that cooling of the sample occurs as heat is transferred from the sample container. Various modes of heat transfer can be used in practicing the scope of the invention, for example, convection heat transfer (forced or passive) and/or conduction heat transfer. As described, sample temperature is monitored by measuring the resistance of the sample. Therefore, both the means for heating and the means for measuring the temperature are preferably provided by the same simple electronic circuit. It will be appreciated that the invention's method of temperature modification and control is especially useful when rapid changes in sample temperature are desired and when small samples are used. Moreover, the invention has particular advantages if electronic control is desired (for example, on a silicon chip).

The invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

All references are expressly incorporated herein by reference.

What is claimed is:

1. A device for controlling the temperature of a liquid sample comprising:
a holder for a liquid sample;
a first and a second electrode positioned to be in electrical contact with said liquid sample when present in said holder; and
a circuit for adjusting the current flowing through the liquid sample to regulate the temperature thereof.

2. A device according to claim 1 wherein said sample holder comprises a capillary tube.

3. A device according to claim 2 wherein said capillary tube is electrically connected to said first and said second electrodes via a conductive joint comprising a semipermeable membrane.

4. A device according to claim 1 wherein said sample holder comprises a chamber of a microfluidic device.

5. A device according to claim 1 wherein said first and said second electrodes are connected to an alternating current power supply.

6. A device according to claim 1 wherein said first and said second electrodes are respectively positioned in a first fluid reservoir and a second fluid reservoir, said reservoirs being positioned to be in fluid and electrical connection with said sample when present.

7. A device according to claim 6 wherein said sample holder is a microfluidic reaction chamber having a cross sectional dimension of between 0.1 and 1000 μm.

8. A device according to claim 1 wherein said circuit for adjusting the current flowing through said sample monitors the electrical resistance of the sample and adjusts the current flowing through the sample in accordance with a time/temperature profile.

9. A device according to claim 1 further comprising a heat sink for cooling said sample.

10. The device according to claim 9 wherein said heat sink comprises a gas in thermal contact with said at least one outer surface of said sample holder.

11. The device according to claim 9 wherein said heat sink is a liquid in thermal contact with said sample holder

12. A method for thermal cycling a liquid sample comprising the steps of:
contacting a liquid sample with first and second electrodes;
applying an electrical current across said electrodes and through said sample; and
adjusting the current flowing through the biological sample to control the temperature of said liquid sample.

13. The method of claim 12 further comprising cooling the liquid sample in accordance with a time/temperature profile.

14. A method for measuring the temperature of a liquid in a conduit comprising measuring the resistance of said liquid and comparing said resistance to a predetermined correlation between resistance in temperature to provide an indication of the temperature of said liquid.

15. A device for controlling the temperature of a liquid sample comprising:
a holder for a liquid sample;
a first and a second electrode positioned to be in electrical contact with said liquid sample when present in said holder; and
a circuit for measuring the resistance of said liquid wherein said resistance is compared to a predetermined correlation between resistance and temperature to provide an indication of the temperature of said liquid.

16. The device of claim 15 wherein said resistance of said liquid is used in a control circuit to regulate the temperature of said liquid by adjusting the current flowing through said liquid sample.
